(19) United States
(12) Patent Application Publication
(10) Pub. No.: US 2026/0215827 A1
(43) Pub. Date: Jul. 30, 2026

(54) Transverse Bone Transport System and Related Method for Transverse Bone Transport

(71) Applicants: ORTHOFIX S.R.L., Verona (IT); TEXAS SCOTTISH RITE HOSPITAL FOR CHILDREN, Dallas, TX (US)

(72) Inventors: Mikhail L. Samchukov, Coppell, TX (US); Andrea Ottoboni, Giacciano con Baruchella (RO) (IT); Davide Gaspari, Villafranca di Verona (VR) (IT); Daniele Venturini, Povegliano Veronese (VR) (IT); Alexander M. Cherkashin, Flower Mound, TX (US); John D. Ross, Ovilla, TX (US); Karen D. Standefer, Flower Mound, TX (US)

(21) Appl. No.: 19/429,310

(22) Filed: Dec. 22, 2025

Related U.S. Application Data
(60) Provisional application No. 63/751,137, filed on Jan. 29, 2025.

(51) Int. Cl. A61B 17/80; A61B 17/00; A61B 17/56; A61B 17/68

(52) U.S. Cl. CPC A61B 17/8004; A61B 2017/00398; A61B 2017/564; A61B 2017/681

(57) ABSTRACT

A transverse bone transport system 1, comprising: a base plate 2 configured to contact an attachment surface of a bone B of a patient and to be securely attached thereto; a movable piece 3 configured to be securely attached to a bone transport segment T, the movable piece 3 being transversally movable relative to the base plate 2; and a distraction device configured to displace the movable piece from a first position to a second position, wherein, in the second position, the movable piece is offset further from the attachment surface compared to the first position.

### CROSS-REFERENCE TO RELATED APPLICATIONS

The present application claims the benefit of U.S. Provisional Application No. 63/751,137, filed Jan. 29, 2025, the entire disclosure of which is hereby incorporated herein by reference.

### FIELD OF APPLICATION

The present invention relates to the general technical field of orthopedics.

More in particular, the invention relates to a system for transverse bone transport and to a method for transverse bone transport employing said system.

### Prior Art

Foot ulcers are a prevalent issue among patients with poorly controlled diabetes or peripheral arterial disease. Transverse bone transport, also referred to as lateral tibial bone transport, is a therapeutic approach for treating these ulcers. This technique involves cutting a segment of bone in the affected leg, often the tibia, and moving it medially or transversely to the bone's anatomical axis. This procedure, commonly associated with the Ilizarov technique, applies continuous tension to the bone callus tissues. This tension can stimulate the growth of new blood vessels in the tissues between the transported bone segment and the base portion, thereby enhancing blood circulation and promoting active neovascularization in the bone and the surrounding soft tissues.

This process can significantly improve the healing of diabetic and peripheral arterial disease-related foot ulcers in the lower extremity.

Currently available transverse bone transport systems typically consist of an external fixation device, distractors, and pins or wires that secure the bone segment to the device. These systems are designed to gradually move a segment of bone laterally or transversely to the longitudinal axis of the limb, facilitating neovascularization and healing in conditions such as diabetic foot ulcers. The fixation device is usually mounted externally, with pins or wires passing through the skin and soft tissues into the bone. Gradual adjustments to the frame by distractors apply controlled tension to the bone and surrounding soft tissues, promoting tissue regeneration and vascular growth.

While effective, these systems can be cumbersome for surgeons to apply, uncomfortable for patients to adjust, and pose a higher risk of infection due to the external hardware.

The technical problem underlining the present invention is that of providing new systems and methods for transverse bone transport overcoming the drawbacks affecting the prior art solutions.

### SUMMARY OF THE INVENTION

The above-mentioned technical problem is solved by a transverse bone transport system, comprising:
a base plate configured to contact an attachment surface of a bone of a patient and to be securely attached thereto;
a movable piece configured to be securely attached to a bone transport segment, the movable piece being transversally movable relative to the base plate; and
a distraction device configured to displace the movable piece from a first position to a second position, wherein, in the second position, the movable piece is offset further from the attachment surface compared to the first position.

In an exemplary embodiment, the bone can be a long bone. However, the bone can also be a different bone, such as a pelvic bone.

Preferably, the movable piece is displaceable in a direction which is substantially orthogonal to the plane of the base plate.

Preferably, the base plate is attached to the bone site by first bone screws, preferably a pair of bone screws.

Preferably, the movable piece is attached to the bone transport segment by second bone screws, preferably a pair of bone screws. Preferably, said second bone screws are inclined inwardly in order to enhance the stability of the connection between the movable piece and the bone transport segment.

Preferably, the base plate comprises an aperture, which can be either a through hole or a C-shaped aperture.

Preferably, the movable piece is at least partially housed in the aperture when in its first position and is lifted above said aperture when in its second position.

The distraction device can be advantageously configured to ensure that the movable piece remains consistently parallel to the base plate throughout its motion from the first position to the second position. Preferably, a mechanical guiding system is employed to achieve said goal.

In the preferred embodiment, the distraction device comprises at least one raising member coupled to the movable piece and at least one actuation system operatively connected to the at least one raising member.

Preferably, the raising members are at least two raising members, each coupled to the opposite end of the movable piece.

In an embodiment, the actuation system comprises at least one linear actuator and the raising members are bars, each hinged at a first end to a corresponding end of a movable piece and at a second end to the at least one linear actuator.

Preferably, two pairs of parallel bars are provided at the opposite ends of the movable piece.

The at least one linear actuator can comprise at least one motor, two drive screws and two nut sliders, the at least one motor being operatively coupled to the drive screws configured to move the nut sliders in opposite directions, the nut sliders being hinged to the second ends of the bars.

In an embodiment, at least one of the bars comprises a lateral pin which is slidingly accommodated within a guiding groove integral with the base plate, to ensure that the movable piece remains consistently parallel to the base plate throughout its motion from the first position to the second position.

In an embodiment, the sliding groove is curvilinear.

Preferably, both lateral bars at the opposite ends of the movable piece have a lateral pin housed in a different guiding grooves.

In a different embodiment, the system can comprise a parallelogram linkage configured to guide the motion of at least one of the bars, to ensure that the movable piece remains consistently parallel to the base plate throughout its motion from the first position to the second position.

Preferably, the parallelogram linkage is defined by the bar, a portion of the movable piece, an auxiliary bar parallel to the bar, and a guiding bar sliding parallel to the movable piece.

Preferably, there are two parallelogram linkages counterposed at both ends of the movable piece.

Instead of a worm screw as described above, other types of linear actuators can be alternatively employed, such as, for example, a rack-and-pinion actuator, a ratcheting rotational solenoid, a cylindrical cam, or the like.

In some embodiments, the actuation system can be advantageously arranged within the base plate, located laterally adjacent to the previously-defined aperture.

In an embodiment, the actuation system comprises driving nuts and the raising members are screws engaged within the driving nuts, said screws being substantially orthogonal to the base plate and constrained to the opposite ends of the movable piece.

In said embodiment, the driving nuts can define an external gear which engage with worms or racks to define either a worm gear or a rack-and-pinion connection. The worms can be provided on the opposite ends of coaxial shafts driven by a single motor or by counterposed twin motors.

In an alternative embodiment, the actuation system comprises at least a first pumping unit and the raising members are inflatable bellows which are interposed between the base plate and the movable piece, the first pumping unit being connected via piping to the inflatable bellows and configured to inflate said inflatable bellows.

The inflatable bellows can be inflated by any suitable fluid, such as a saline solution or air.

Preferably, the actuation system further comprises a second pumping unit configured to deflate said inflatable bellows.

Preferably, the two pumping units are external to the patient's body and connected to the base plate via at least one flexible tube of the piping.

Still preferably, the distraction device further comprises return springs configured to return the movable piece in an initial position when the inflatable bellows are deflated.

The transverse bone transport system according to the invention may further comprise a power source for powering said distraction device

The power source may comprise one or more batteries, or else one or more receiving coils configured to receive power from an external primary coil. Other power sources such as one or more capacitors can be employed as an alternative or as a complement.

The power source can be conveniently housed in an inner compartment of the base plate, except in the embodiment comprising external pumping units.

Further solutions can be employed to drive the movable piece with respect to the base plate, such as for instance: a piezoelectric actuator, a linear actuator of any kind, a linear ratcheting with springs, a shape memory actuator or a peculiar compliant plate geometry.

The base plate can conveniently incorporate sensors, such as blood flow sensors.

The above-mentioned technical problem is also solved by a transverse bone transport method, comprising the steps of:
cutting the skin and subcutaneous tissues of a patient to access a bone site;
implanting a base plate with at least an aperture on the bone site;
performing an osteotomy to detach a bone transport segment from the bone site;
anchoring the bone transport segment to a movable piece attached to the base plate;
closing the incision;
after the incision is closed, operating a distraction device configured to displace the movable piece with respect to the base plate.

The method can advantageously employ any of the transverse bone transport systems described above.

Preferably, the movable piece is displaced in a direction which is substantially orthogonal to the base plate.

Preferably, the base plate is implanted by means of self-tapping bone screws.

Preferably, the osteotomy is performed by either a laser cutting system, an oscillating saw or an osteotome.

The method can employ an aperture of the base plate as a guide for the osteotomy.

If external pumping units are adopted, a flexible tube for connecting the external pump units to inflatable bellows interposed between the base plate and the movable piece is routed through the incision.

Further features and advantages will be more apparent from the following detailed description of preferred non-limiting embodiments of the present invention, and from the dependent claims which outline preferred and particularly advantageous embodiments of the invention.

### BRIEF DESCRIPTION OF THE DRAWINGS

The accompanying drawings illustrate implementations of the systems, devices, and methods disclosed herein and together with the description, serve to explain the principles of the present disclosure.

FIG. 1 shows a perspective view of a first embodiment of the transverse bone transport system according to the invention;

FIG. 2 shows a view from above of the first embodiment of the transverse bone transport system according to the invention;

FIG. 3 shows a perspective view of the first embodiment of the transverse bone transport system with bone screws attached, in a first configuration;

FIG. 4 shows a different perspective view of the first embodiment of the transverse bone transport system with bone screws attached, in a second configuration;

FIG. 5 shows a perspective view of the first embodiment of the transverse bone transport system anchored to a bone site, in its first configuration;

FIG. 6 shows a perspective view of the first embodiment of the transverse bone transport system anchored to a bone site, in its second configuration;

FIG. 7 shows a perspective view of the first embodiment of the transverse bone transport system, with the base plate omitted to reveal the internal components;

FIG. 8 shows a different perspective view of the first embodiment of the transverse bone transport system with the base plate omitted;

FIG. 9 shows a top view, sectioned along a longitudinal plane, of the first embodiment of the transverse bone transport system;

FIG. 10 shows a lateral view from behind of the first embodiment of the transverse bone transport system;

FIG. 11 shows a perspective view of a second embodiment of the transverse bone transport system according to the invention in a first configuration, with a removable cover omitted to reveal the interior of an upper compartment;

FIG. 12 shows a perspective view from a different side of the second embodiment of the transverse bone transport system according to the invention in a second configuration;

FIG. 13 shows a perspective exploded view of the second embodiment of the transverse bone transport system according to the invention;

FIG. 14 shows a perspective view of the second embodiment of the transverse bone transport system anchored to a bone site, in its first configuration;

FIG. 15 shows a perspective view of a third embodiment of the transverse bone transport system according to the invention, in a first configuration;

FIG. 16 shows a top view of the third embodiment of the transverse bone transport system according to the invention, with the internal components depicted using shadow lines;

FIG. 17 shows a perspective view of the third embodiment of the transverse bone transport system according to the invention, in a second configuration;

FIG. 18 shows a perspective view of the third embodiment of the transverse bone transport system according to the invention anchored to a bone site, in its first configuration;

FIG. 19 shows a perspective view of a fourth embodiment of the transverse bone transport system according to the invention, in a first configuration;

FIG. 20 shows a different perspective view of the fourth embodiment of the transverse bone transport system according to the invention, in its first configuration;

FIG. 21 shows a side view, sectioned along a medial plane, of the fourth embodiment of the transverse bone transport system according to the invention anchored to a bone site, in its first configuration;

FIG. 22 shows a perspective view of the fourth embodiment of the transverse bone transport system according to the invention anchored to a bone site, in its second configuration.

In the different figures, analogous elements will be identified with analogous reference numbers.

Moreover, in the figures, if there is a plurality of elements which are similar to each other, only some of them will be indicated by a reference number for greater clarity; the other similar elements, although not indicated by a suitable reference number, are to be understood as included by analogy.

These figures will be better understood by reference to the following detailed description.

It is noted that, even though the exemplary embodiments shown in the figures are applied to a long bone, the present invention can also be employed with other bones, such as for instance pelvic bones.

### DETAILED DESCRIPTION

While the manufacturing and using of embodiments of the present disclosure is discussed in detail below, it should be appreciated that the present disclosure provides many applicable inventive concepts that can be embodied in a wide variety of specific contexts. The specific embodiments discussed herein are merely illustrative of specific ways to make and use the disclosure and does not delimit the scope of the disclosure.

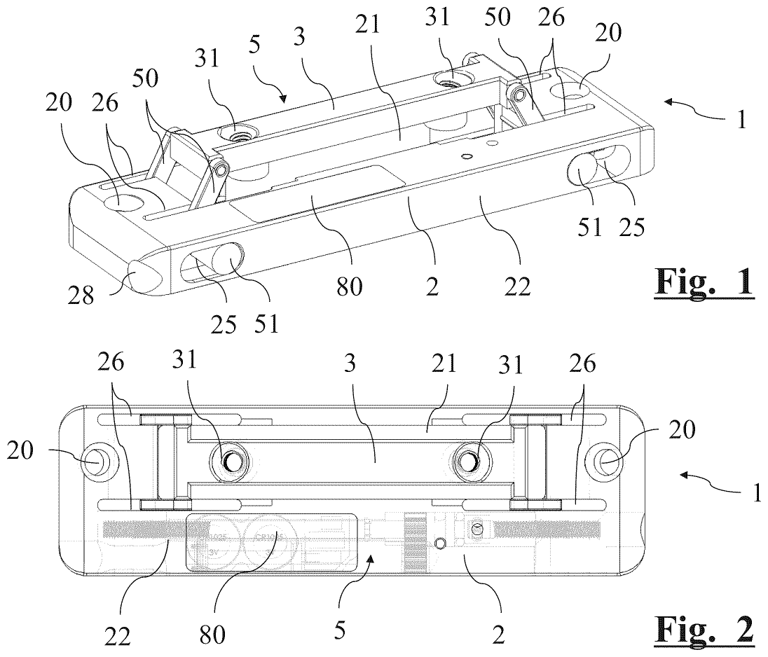

FIG. 1 shows a perspective view of a first embodiment of the transverse bone transport system 1.

The transverse bone transport system 1 comprises a base plate 2 configured to contact a long bone of a patient and to be securely attached thereto.

The base plate 2 is substantially planar with an elongated shape, preferably rectangular. A bottom surface is designed to contact the patient's bone, while a top surface is positioned opposite.

The base plate 2 comprises an elongated aperture 21 that extends through the plate, spanning from its bottom surface to its top surface. Both the base plate 2 and the elongated aperture 21 extend along the same longitudinal direction. The elongated aperture 21 is configured to house a movable piece 3, which has a correspondingly elongated shape, in a first position thereof. The elongated aperture 21 comprises, at both ends thereof, two lateral slits 26, separated by a solid portion, which extend in the longitudinal direction toward the opposite ends of the base plate 2.

The base plate 2 comprises a plurality of first through holes 20 for bone screws. In the depicted embodiment, the first through holes 20 are two in number and positioned next to the opposite ends of the elongated aperture 21. The first through holes 20 are slightly inclined with respect to a transverse direction orthogonal to the planar shape of the base plate 2. The first through holes 20 are preferably non-threaded.

The base plate 2 comprises a lateral casing 22 positioned adjacent to the elongated aperture 21. The lateral casing 22 comprises, in its upper portion, an upper compartment sealed by a removable cover 80. The removable cover 80 impermeably seals the upper compartment.

The base plate 2 further comprises, at the opposite ends of its lateral face next to the lateral casing 22, two slots 25 which extend in the longitudinal direction and communicate with the hollow portion.

The movable piece 3 is also substantially planar with an elongated shape, preferably rectangular. Its length is preferably between 50 mm and 90 mm, while its width is preferably between 10 mm and 20 mm.

Along its length, the movable piece 3 comprises a plurality of second through holes 31. Preferably, the movable piece 3 has two second through holes 31 that are slightly inclined relative to the transverse direction and spaced apart by a distance preferably of 30 to 50 mm. Preferably, the second through holes are threaded.

The movable piece 3, as further discussed below, has a bottom surface which is configured to contact a bone transport segment of the patient and to be securely attached thereto via bone screws inserted in the second through holes 31.

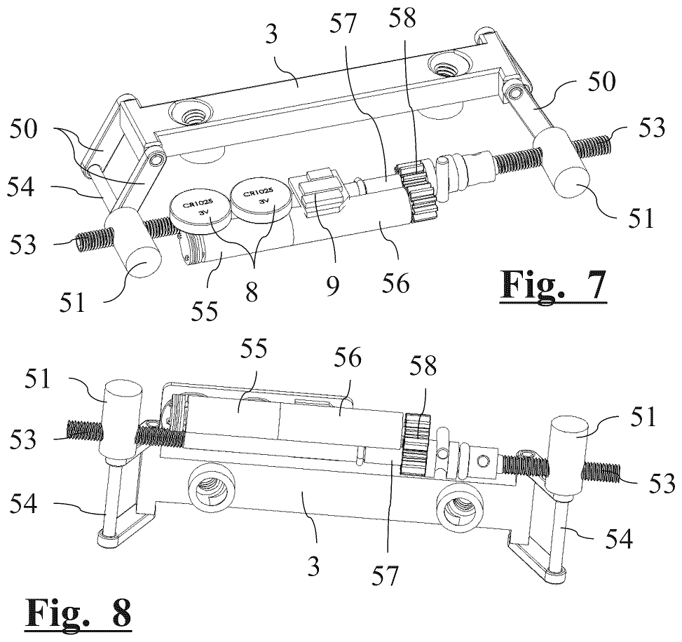

The transverse bone transport system 1 comprises a distraction device 5, which is better described in the following with reference to FIGS. 7 and 8.

The distraction device 5 makes it possible to displace the movable piece 3 from a first position depicted in FIG. 2 to a second position depicted in FIG. 1, and the other way around. In the first position, the movable piece 3 is housed within the elongated aperture 21; in the second position, the movable piece 3 is extracted and lifted above the elongated aperture 21.

It is observed that the movable piece 3 remains consistently parallel to the base plate 2

### Throughout its Motion From the First Position to the Second Position.

The distraction device 5 comprises a scissor jack mechanism having at least two opposite pairs of bars 50, which pair of bars 50 are hinged to the respective opposite ends of the movable piece 3. The opposite ends of the two pair of bars are coupled to two nut sliders 51, which are moved in opposing directions by respective drive screws. The two nut sliders 51 slide within the previously described lateral slots 25.

### The Bars 50 Function As Raising Members for the Movable Piece 3.

In the initial position of the movable piece 3, the bars 50 are inclined upwards at a first angle relative to the base plate 2 and are preferably positioned within the lateral slits 26. This initial inclination is sufficiently steep, preferably between 5° and 30°, with an ideal range of 10° to 17°, minimizing the axial force required to elevate the bar mechanism. Upon activation of the distraction device 5, the nut sliders 51 move closer together, increasing the inclination of the bars and causing the movable piece 3 to ascend toward the second position. In this second position, the bars 50 achieve a steeper upward inclination, ideally between 60° and 80°, with a preferred angle of 70°.

The length of the bars 50 is preferably between 5 mm and 30 mm, still preferably between 12 mm and 18 mm.

A reverse operation can also be performed to return the movable piece 3 to its position within the elongated aperture 21.

FIG. 2 shows a view from above of the first embodiment of the transverse bone transport system 1.

In the figure, it is possible to appreciate that most of the distraction device, and specifically the drive screws, are in the lateral casing 22, which is adjacent to the elongated aperture 21 dedicated to the movable piece 3.

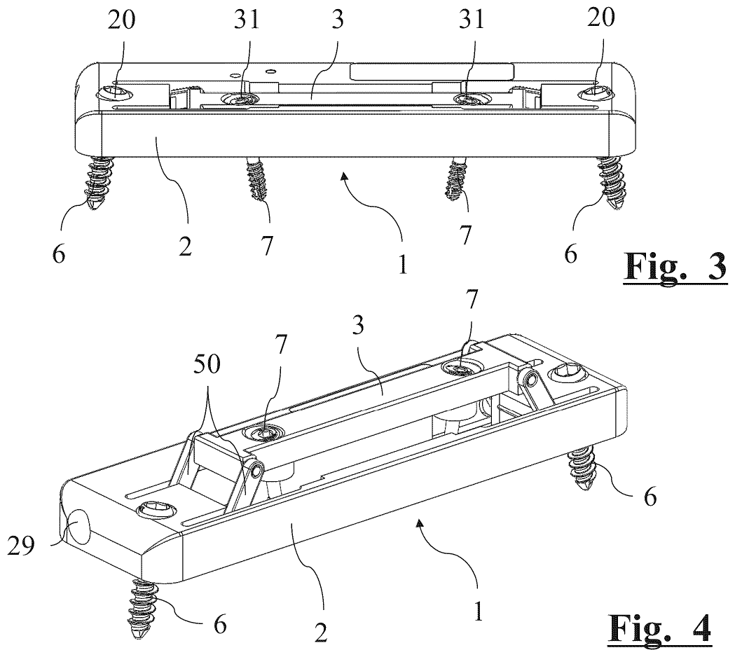

FIG. 3 shows a perspective view of the first embodiment of the transverse bone transport system 1 with bone screws attached, in the first configuration.

The figure shows two first bone screws 6 inserted in the first through holes 20 and two second bone screws 7 inserted in the second through holes 31.

The first bone screws 6 preferably have a larger diameter compared with the second bone screws 7.

As shown in the figure, due to the inclination of the through holes 20, 31, the bone screws 6, 7 are angled relative to the transverse direction. In particular, the first bone screws 6 are directed outwardly, while the second bone screws 7 are directed inwardly along a transversal plane oriented along the longitudinal extension of the base plate 2. The angle of the first bone screws 6 relative to the transverse direction ranges from 10° to 30°, with a preferred angle of 20°. The angle of the first bone screws 7 relative to the transverse direction ranges from 10° to 30°, with a preferred angle of 20°.

FIG. 4 shows a perspective view of the first embodiment of the transverse bone transport system 1 with bone screws attached, in the second configuration.

The figure shows that, via the second screws 7, the transverse bone transport system 1 raises a bone transport segment away from its original location on the bone.

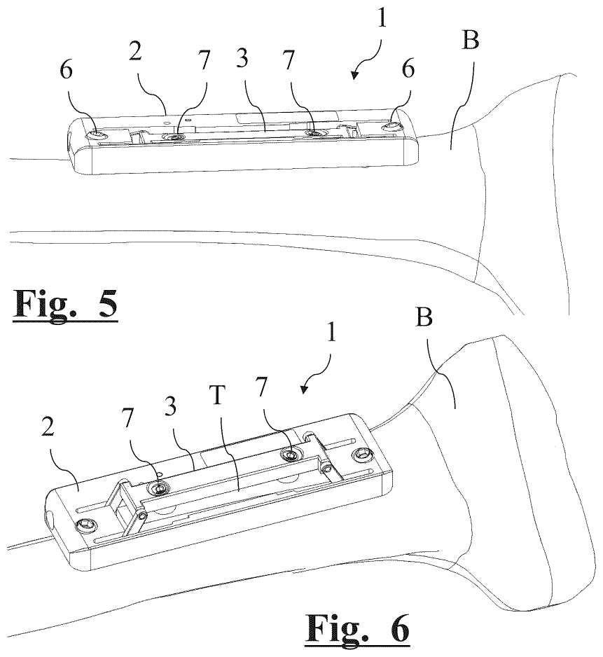

FIG. 5 shows a perspective view of the first embodiment of the transverse bone transport system 1 anchored to a bone site, in its first configuration.

The figure shows how, in a first step of a method for bone transport, the transverse bone transport system 1 is anchored to a long bone B, in particular a tibia. In the same step, a bone transport segment is also cut from the long bone and anchored below the movable piece 3 by means of the second bone screws 7.

It is observed that the base plate 2 is preferably attached, via the first bone screws 6, with its longitudinal axis aligned parallel to the long bone B.

FIG. 6 shows a perspective view of the first embodiment of the transverse bone transport system 1 anchored to a bone site, in its second configuration.

The figure shows how, in a second step of a method for transverse bone transport, the movable piece 3 is progressively moved toward its second position, therefore raising the bone transport segment T which is attached to the movable piece via the second bone screws 7.

FIG. 7 shows a perspective view of the first embodiment of the transverse bone transport system 1, with the base plate omitted to reveal the internal components.

The transverse bone transport system 1 comprises batteries 8, specifically two button cells, housed within the upper compartment and serving as the power source for the distraction device 5.

The transverse bone transport system 1 further comprises a control unit 9, preferably in the form of one or more PCBs, which is preferably also housed in the upper compartment.

The distraction device 5 comprises an electric motor 55 and a gearbox 56 operatively connected to the electric motor 55. Preferably, both the electric motor 55 and the gearbox 56 have a cylindrical shape, with the two cylinders being coaxial and having the same diameter. The electric motor 55 and the gearbox 56 may be connected via a magnetic coupling.

The distraction device 5 further comprises a shaft 57, which defines guide screws 53 at its respective free ends. The two guide screws 53 are threaded in opposite directions. The shaft 57 is parallel to the common axis of the electric motor 55 and the gearbox 56 and is positioned between the electric motor 55 and the elongated aperture 21.

The gearbox 56 and the shaft 57 are operatively connected by a gearing 58, which features two gears, preferably of the same diameter.

The nut sliders 51 have an internally threaded hole and are engaged with the two guide screws 53. Each nut slider 51 includes a lateral axle 54 on which two of the previously described bars 50 are hinged. The nut sliders 52 are guided within the lateral slots 25.

The control unit 9 activates the electric motor 55, which is powered by the batteries 8. The electric motor 55 rotates the shaft 57 through the gearbox 56 and the gearing 58. The guide screws 53 at both ends of the shaft 57 move the nut sliders 51, which, in turn, shift the bottom ends of the bars, thereby raising or lowering the movable piece 3.

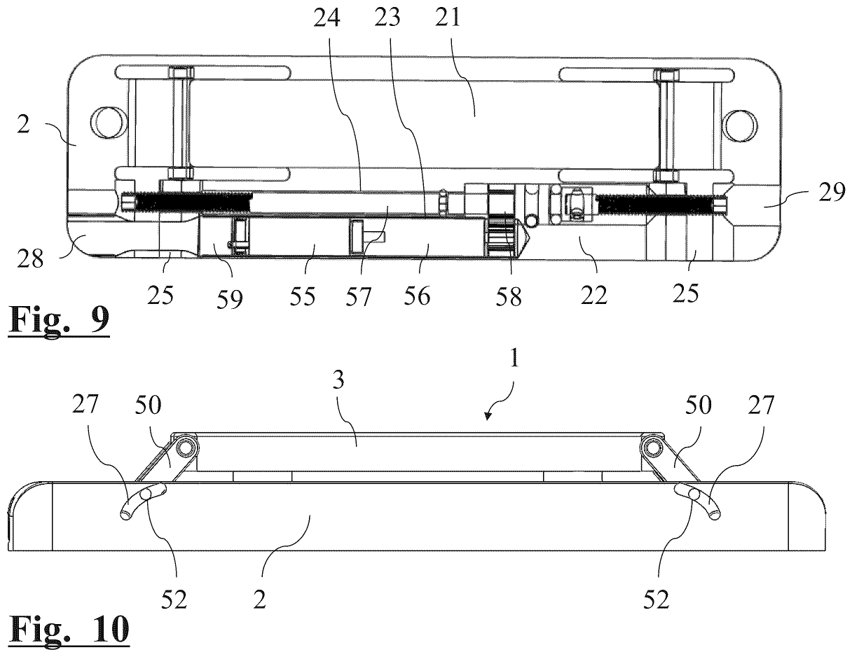

FIG. 9 shows a top view, sectioned along a longitudinal plane, of the first embodiment of the transverse bone transport system 1.

The figure illustrates that the lateral casing 22 comprises a first tubular cavity 23 and a second tubular cavity 24, both preferably cylindrical in shape. The first tubular cavity 23, located laterally, features a first inlet 28 that opens on a proximal or distal side of the base plate 2. The second tubular cavity 24, positioned between the first tubular cavity 23 and the elongated aperture 21, features a second inlet 29 that opens on the side of the base plate 2 opposite to the first inlet 28.

The electric motor 55 and gearbox 56 are inserted in the first tubular cavity 23 through the first inlet 28, with the electric motor 55 being proximal to the first inlet 28. The first tubular cavity 56 is closed with an impermeable plug 59 which seals the cavity.

The shaft 57 is inserted in the second tubular cavity 24 through the second inlet 29.

O-rings are conveniently positioned on both sides of the gearing 58 to ensure watertightness, along with the removable cover 24 and the impermeable plug 59, for the entire volume enclosing the batteries 8, the control unit 9, the electric motor 55 and the gearbox 56. As a result, all the electronics and electronic components in the implantable device are encased within a watertight envelope, effectively isolating them from the patient's body fluids.

FIG. 10 shows a lateral view from behind of the first embodiment of the transverse bone transport system 1.

The figure illustrates that the base plate 2 comprises one or more guide grooves 27 designed to slidingly accommodate corresponding lateral pins 52, which are integral with corresponding bars 50. These guide grooves 27 direct the lateral pins 52 along a specific path, ensuring that the bar mechanism, formed by the bars 50 and the movable piece 3, follows the intended trajectory. Ideally, this trajectory ensures that the movable piece 3 remains parallel to the base plate 2 at all times.

In the specific embodiment shown in the figure, there are two guide grooves 27, positioned on a lateral wall of the base plate 2 adjacent to the elongated aperture 21, opposite the side where the distraction device 5 is located. The guide grooves 27 follow a curvilinear path with their concavity facing the bottom surface of the base plate 2. Preferably, these guide grooves 27 are designed as through cuts.

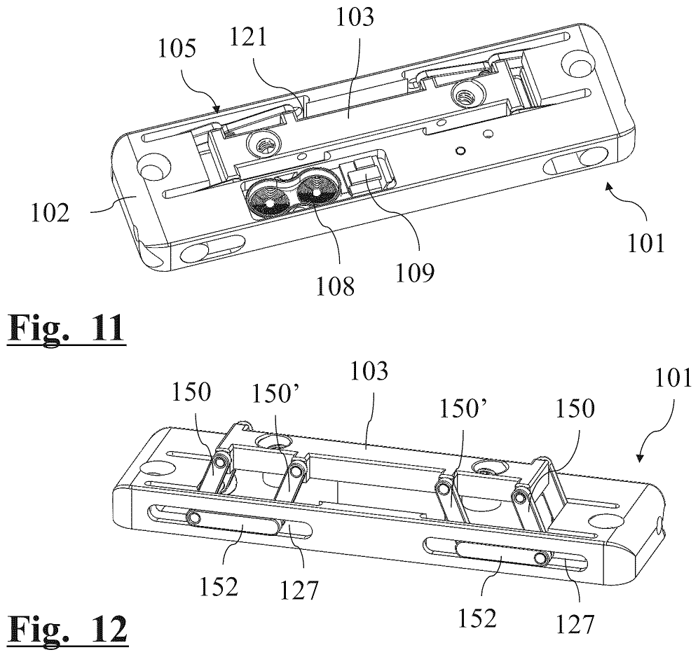

FIG. 11 shows a perspective view of a second embodiment of the transverse bone transport system 101 according to the invention. The transverse bone transport system 101 is depicted in a first configuration, with a removable cover omitted to reveal the interior of an upper compartment.

The second embodiment is noted to share the majority of its structural features with the previously described first embodiment. Therefore, only the differing features will be detailed below, with reference to the earlier description for the features common to both embodiments.

The transverse bone transport system 101 according to the second embodiment also comprises a base plate 102, a movable piece 103 and a distraction device 105 configured to displace the movable piece 103 with respect to the base plate 102.

The base plate 102 is configured to be anchored to a bone site while the movable piece 103 is configured to be attached to a bone transport segment.

As in the first embodiment, the transverse bone transport system 101 transitions between two configurations. In the first configuration, the movable piece 103 is in a first position, partially inserted within an elongated aperture 121 of the base plate. In the second configuration, the movable piece 103 is in a second position, fully extracted and raised above the elongated aperture 121.

Similar to the first embodiment, the base plate 102 includes an upper compartment that houses a power source and a control unit 109. However, unlike the first embodiment, the power source consists of one or more receiving coils 108. These receiving coils are specifically designed to receive induced power from a primary coil located outside the patient's body as part of a wireless power transfer system.

It is noted that the receiving coils 108 can either replace or complement the batteries used in the first embodiment, while the batteries can replace or complement the receiving coils in this second embodiment.

FIG. 12 shows a perspective view from a different side of the second embodiment of the transverse bone transport system according to the invention in a second configuration, wherein the movable piece is raised.

The figure illustrates the raising members, represented by the bars 150, which are analogous to those in the first embodiment. However, in this second embodiment, the mechanism's path is not guided by a lateral pin sliding within a guide groove. Instead, it is directed by at least one parallelogram linkage, preferably two parallelogram linkages, as detailed below.

Each parallelogram linkage comprises an auxiliary bar 150′ and a guiding bar 152. The auxiliary bar 150′ has the same length as, and is parallel to, one of the bars 150. It is hinged to the movable piece 103 at a point offset from the bar 150. The guiding bar 152 connects with hinges the lower ends of the auxiliary bar 150′ and its corresponding bar 150 and slides within a horizontal guide groove 127 located on the side of the base plate 102. Together, the bar 150, auxiliary bar 150', movable piece 103, and guiding bar 152 form the parallelogram linkage. This configuration ensures that the movable piece 103 remains substantially parallel to the base plate 102 as it moves between the first and second positions.

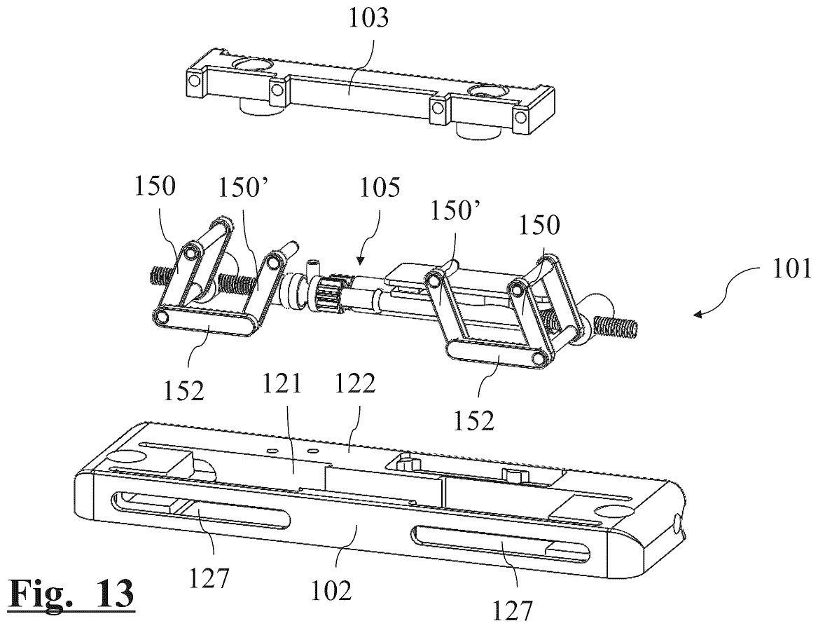

FIG. 13 shows a perspective exploded view of the second embodiment of the transverse bone transport system according to the invention.

The figure shows the details of the distraction device 105 with the two counterposed parallelogram linkage both defined by a bar 150, an auxiliary bar 150', part of the movable piece 103, and a guiding bar 152.

The figure also shows that the motor with the guide screws of the distraction device 105 are housed, as in the first embodiment, in a lateral casing 122 of the base plate 102, next to the elongated aperture 121. The base plate 102 features on its side wall the two horizontal guide grooves 127, which are aligned with each other.

It is noted that both the parallelogram linkages and the lateral pin sliding in the guide groove serve the same function. However, the mechanisms are not mutually exclusive and they can be integrated within a same embodiment.

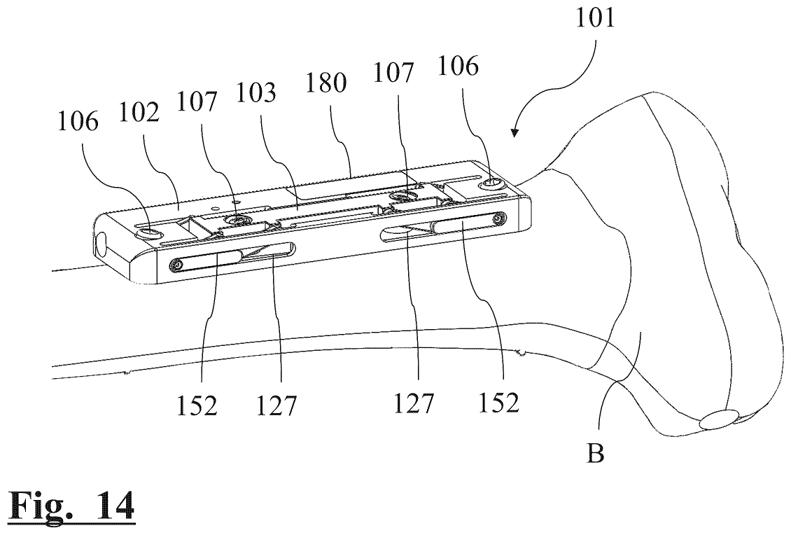

FIG. 14 shows a perspective view of the second embodiment of the transverse bone transport system 101 anchored to a bone site, in its first configuration.

The figure shows how, in a first step of a method for bone transport, the transverse bone transport system 1 is anchored to a long bone B, in particular a tibia. In the same step, a bone transport segment is also cut from the long bone and anchored below the movable piece 103 by means of the second bone screws 107.

It is observed that the base plate 102 is preferably attached, via the first bone screws 106, with its longitudinal axis aligned parallel to the long bone B.

The figure also shows the end position of the guiding bars 152 within the horizontal guide grooves 127 in the first configuration.

The figure further shows a removable cover 180 that seals the upper compartment housing the power source and the control unit.

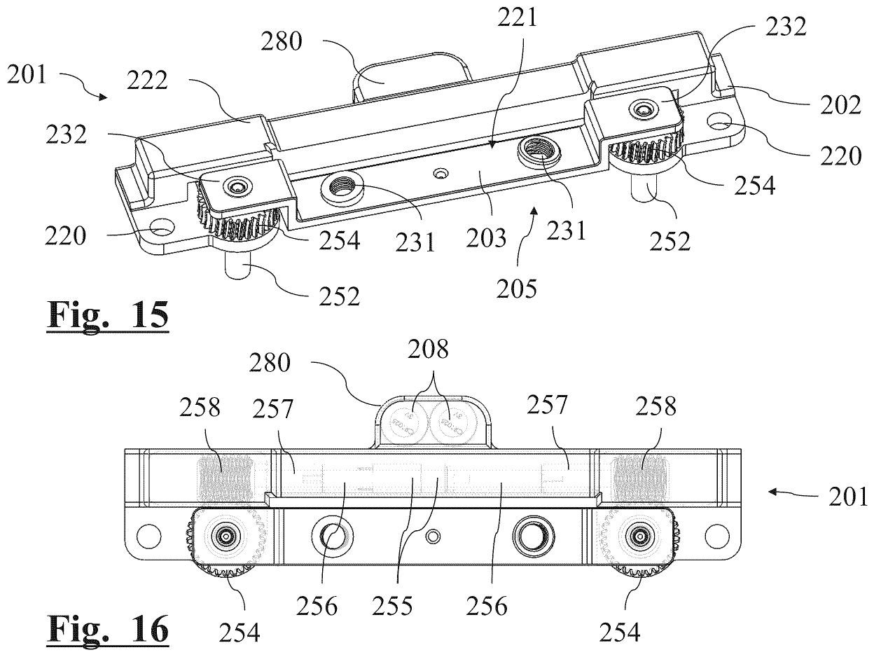

FIG. 15 shows a perspective view of a third embodiment of the transverse bone transport system 201 according to the invention, in a first configuration.

The transverse bone transport system 201 according to the third embodiment also comprises a base plate 202, a movable piece 203 and a distraction device 205 configured to displace the movable piece 203 with respect to the base plate 202.

The base plate 202 is configured to be anchored to a bone site while the movable piece 203 is configured to be attached to a bone transport segment.

As in the first two embodiments, the transverse bone transport system 201 transitions between two configurations. In the first configuration, the movable piece 203 is in a first position, partially inserted within a lateral, C-shaped aperture 221 of the base plate 202. In the second configuration, the movable piece 203 is in a second position, fully extracted and raised above the C-shaped aperture 221.

The base plate 202 comprises a plurality of first through holes 220 for bone screws. In the depicted embodiment, the first through holes 220 are two in number and positioned next to the opposite ends of the C-shaped aperture 221. As in the first two embodiments, the first through holes 220 can be slightly inclined. The first through holes 220 are preferably non-threaded.

The base plate 202 comprises a lateral casing 222 positioned adjacent to the C-shaped aperture 221. The lateral casing 222 comprises, on a side opposed to the C-shaped aperture 221, a sealed lateral compartment 280.

The movable piece 203 is also substantially planar with an elongated shape, preferably rectangular. It has a central lowered portion and two raised end portions 232.

In its central lowered portion, the movable piece 203 comprises a plurality of second through holes 231. Preferably, the movable piece 203 has two second through holes 231 that are slightly inclined as in the first two embodiments. Preferably, the second through holes are threaded.

The transverse bone transport system 201 comprises a distraction device 205, which is better described in the following with reference to FIG. 16.

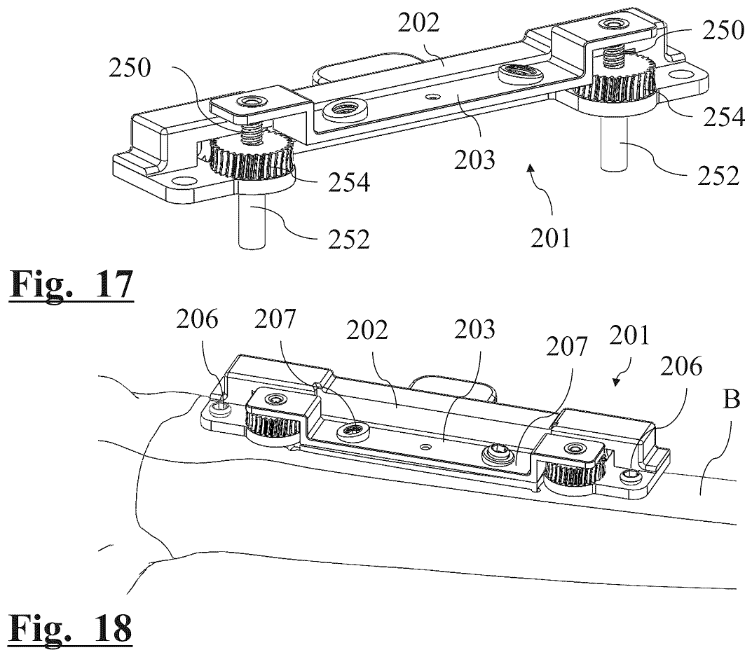

The distraction device 205 comprises two vertical tubular elements 252 housing vertical screws 250, which are visible in FIG. 17. The vertical screws are attached to the raised end portions 232 of the movable piece 203. The distraction device 205 further comprises two driving nuts 254 with an external gear and an internal thread. The vertical screws engage with the internal threads of the two driving nuts 254.

FIG. 16 shows a top view of the third embodiment of the transverse bone transport system 201 according to the invention, with the internal components depicted using shadow lines.

The transverse bone transport system 201 comprises batteries 208, specifically two button cells, housed within the lateral compartment 280 and serving as the power source for the distraction device 205.

The batteries can be substituted or complemented by one or more receiving coils as in the second embodiment.

The distraction device 205 comprises an electric motor or two electric motors 255 facing in different directions, and gearboxes 256 operatively connected to them. Preferably, the electric motors 255 and the gearboxes 256 have a cylindrical shape, are coaxial and have the same diameter. The gearboxes 256 are connected to opposite shafts 257 which, at their ends, feature two worms 258. The worms 258 engage in a worm gear with the previously described external gear of the driving nuts 254 and set them into rotation upon activation of the electric motor(s) 255.

FIG. 17 shows a perspective view of the third embodiment of the transverse bone

### Transport System 201 in its Second Configuration.

As can be seen, in this second configuration, the rotation of the driving nuts 254 has caused the vertical screws 250 to rise within the vertical tubular elements 252, thereby lifting the entire movable piece 203. Since the driving gears 258 are rotated by the same amount, the movable piece 203 is maintained substantially parallel to the base plate 202.

It is observed that the vertical screws 250 function as raising members for the movable piece 203.

FIG. 18 shows a perspective view of the third embodiment of the transverse bone transport system 201 according to the invention anchored to a bone site B, preferably a long bone such as a tibia.

The figure shows that the base plate 202 is anchored to the bone site B via first bone screws 206, while second bone screws 207 anchor the bone transport segment to the movable piece 203.

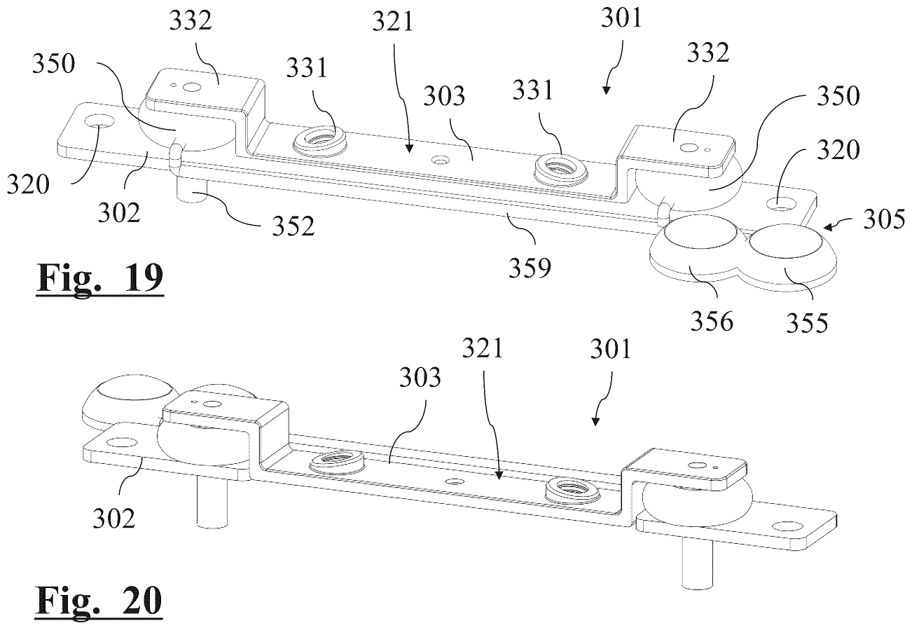

FIG. 19 shows a perspective view of a fourth embodiment of the transverse bone transport system 301 according to the invention, in a first configuration.

As in the previous embodiment, the transverse bone transport system 301 according to the fourth embodiment also comprises a C-shaped base plate 302, an elongated movable piece 303 and a distraction device 305 configured to displace the movable piece 303 with respect to the base plate 302.

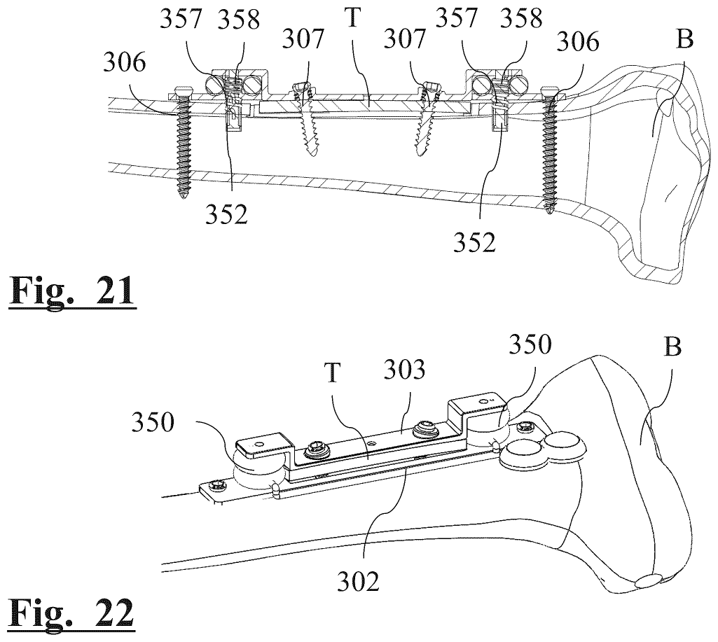

As better depicted in FIGS. 20 and 21, the base plate 302 is configured to be anchored to a bone site B while the movable piece 303 is configured to be attached to a bone transport segment T.

As in the other embodiments, the transverse bone transport system 301 transitions between two configurations. In the first configuration, the movable piece 303 is in a first position, partially inserted within a lateral, C-shaped aperture 321 of the base plate 302. In the second configuration, the movable piece 303 is in a second position, fully extracted and raised above the C-shaped aperture 321.

The base plate 302 comprises a plurality of first through holes 320 for bone screws. In the depicted embodiment, the first through holes 320 are two in number and positioned next to the opposite ends of the C-shaped aperture 321. As in the other embodiments, the first through holes 320 can be slightly inclined. The first through holes 320 are preferably non-threaded.

The movable piece 303 is also substantially planar with an elongated shape, preferably rectangular. It has a central lowered portion and two raised end portions 332.

In its central lowered portion, the movable piece 303 comprises a plurality of second through holes 331. Preferably, the movable piece 303 has two second through holes 331 that are slightly inclined as in the first two embodiments. Preferably, the second through holes are threaded.

### The Transverse Bone Transport System 301 Comprises a Distraction Device 305.

The distraction device 305 comprises two vertical tubular elements 352 housing vertical posts 357, which are visible in FIG. 21. The upper ends of the vertical posts are attached to the raised end portions 332 of the movable piece 303. The vertical posts 357 can slide within the tubular elements 352 along their vertical axis, which is substantially perpendicular to the plane of the base plate 302. As they move along this axis, the vertical posts 357 either raise or lower the movable piece 303.

The distraction device 305 further comprises two inflatable bellows 350 which are interposed between the base plate 302 and the two raised end portions 332 of the movable piece 303. The distraction device 305 further comprises a first pump unit 355, a second pump unit 356, and piping 359 that connects both pump units 355 and 356 to the inflatable bellows 350.

The first pump unit 355 is designed to pump a fluid, such as a saline solution or air, into both inflatable bellows 350 simultaneously. Conversely, the second pump unit 356 is configured to simultaneously extract the fluid from the inflatable bellows 350. For convenience, the pump units 355 and 356 are positioned outside the patient's body, with the piping 359 preferably consisting of a flexible tube capable of passing beneath the gusset created for the osteotomy.

When the inflatable bellows 350 are inflated, as depicted in FIG. 22, the movable piece 303 with the associated bone transport segment T raises in its second position. When the inflatable bellows 350 are deflated the movable piece 303 can move back to its first position.

It is observed that the inflatable bellows 350 function as raising members for the movable piece 303.

FIG. 20 shows a different perspective view of the fourth embodiment of the transverse bone transport system 301 according to the invention, in its first configuration.

The figure illustrates the open side of the C-shaped aperture 321 in the base plate 302, where the movable piece 303 is housed in the first configuration.

FIG. 21 shows a side view, sectioned along a medial plane, of the fourth embodiment of the transverse bone transport system 301 according to the invention anchored to a bone site, in its first configuration.

The figure illustrates the first bone screws 306 and the second bone screw 307 which respectively anchor the base plate 302 to the bone site B and the movable piece 303 to the bone transport segment T.

The figure further illustrates return springs 358 set around the vertical posts 357 to bring back the movable piece 303 in its first position when the inflatable bellows 350 are deflated.

FIG. 22 shows a perspective view of the fourth embodiment of the transverse bone transport system 301 according to the invention anchored to a bone site, in its second configuration.

It can be observed that the inflatable bellows 350 are inflated, thus raising the movable piece 303 with the associated bone transport segment T above the plane of the base plate 302 and the surface of the bone site B.

The different embodiments depicted above can all be employed in a method for performing transverse bone transport.

The method comprises the following surgical steps.

The surgeon cuts the skin and tissues to access the bone site B, preferably a tibial bone site, employing an open surgery approach.

The base plate 2, 102, 202, 302 is implanted on the bone site B using first bone screws 6, 106, 206, 306. The bone screws can be self-tapping and cannulated in order to use guide wires.

An osteotomy is performed, preferably through a laser cutting system, in order to detach the bone transport segment T from the rest of the bone. This can be done either before or after the implant of the base plate 2, 102, 202, 302. The base plate 2, 102, 202, 302 itself can be used as a template for cutting the bone transport segment T.

The bone transport segment T is then attached to the movable piece 3, 103, 203, 303 through the second bone screws 6, 106, 206, 306.

The incision is then closed. If the fourth embodiment of the transverse bone transport system 301 is employed, care should be taken to route the flexible tube of the piping 359 out of the patient's body.

In a post-operative surgery, the step of moving the movable piece 3, 103, 203, 303 is performed. The movable piece 3, 103, 203, 303 originally in its first position, is activated to reach over time its second position.

It will be understood that particular embodiments described herein are shown by way of illustration and not as limitations of the disclosure. The principal features of this disclosure can be employed in various embodiments without departing from the scope of the disclosure. Those skilled in the art will recognize, or be able to ascertain using no more than routine experimentation, numerous equivalents to the specific procedures described herein. Such equivalents are considered to be within the scope of this disclosure and are covered by the claims.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

All of the methods disclosed and claimed herein can be made and executed without undue experimentation in light of the present disclosure. While the methods of this disclosure have been described in terms of preferred embodiments, it will be apparent to those of skill in the art that variations may be applied to the methods and in the steps or in the sequence of steps of the method described herein without departing from the concept, spirit and scope of the disclosure. All such similar substitutes and modifications apparent to those skilled in the art are deemed to be within the spirit, scope and concept of the disclosure as defined by the appended claims.

1. A transverse bone transport system, comprising:
a base plate configured to contact an attachment surface of a bone of a patient and to be securely attached thereto;
a movable piece configured to be securely attached to a bone transport segment, the movable piece being transversally movable relative to the base plate; and
a distraction device configured to displace the movable piece from a first position to a second position, wherein, in the second position, the movable piece is offset further from the attachment surface compared to the first position.

2. The transverse bone transport system according to claim 1, wherein the base plate comprises an aperture, the movable piece being at least partially housed in the aperture when in its first position and being lifted above said aperture when in its second position.

3. The transverse bone transport system according to claim 1, wherein the distraction device is configured to ensure that the movable piece remains consistently parallel to the base plate throughout its motion from the first position to the second position.

4. The transverse bone transport system according to claim 1, wherein the distraction device comprises at least one raising member coupled to the movable piece and at least one actuation system operatively connected to the at least one raising member.

5. The transverse bone transport system according to claim 4, wherein the raising members comprise at least two raising members, each coupled to an opposite end of the movable piece.

6. The transverse bone transport system according to claim 5, wherein the actuation system comprises at least one linear actuator and the raising members are bars, each hinged at a first end to a corresponding end of a movable piece and at a second end to the at least one linear actuator.

7. The transverse bone transport system according to claim 6, wherein the at least one linear actuator comprises at least one motor, two drive screws and two nut sliders, the at least one motor being operatively coupled to the drive screws configured to move the nut sliders in opposite directions, the nut sliders being hinged to the second ends of the bars.

8. The transverse bone transport system according to claim 4, wherein the actuation system is arranged within the base plate, located laterally adjacent to an aperture of the base plate accommodating the movable piece in its first position.

9. The transverse bone transport system according to claim 6, wherein at least one of the bars comprises a lateral pin which is slidingly accommodated within a guiding groove integral with the base plate.

10. The transverse bone transport system according to claim 6, further comprising a parallelogram linkage configured to guide the motion of at least one of the bars.

11. The transverse bone transport system according to claim 5, wherein the actuation system comprises driving nuts and the raising members are screws engaged within the driving nuts, said screws being substantially orthogonal to the base plate and constrained to the opposite ends of the movable piece.

12. The transverse bone transport system according to claim 5, wherein the actuation system comprises at least a first pumping unit and the raising members are inflatable bellows which are interposed between the base plate and the movable piece, the first pumping unit being connected via piping to the inflatable bellows and configured to inflate said inflatable bellows.

13. The transverse bone transport system according to claim 12, wherein the actuation system further comprises a second pumping unit configured to deflate said inflatable bellows.

14. The transverse bone transport system according to claim 12, wherein the distraction device further comprises return springs configured to return the movable piece in an initial position when the inflatable bellows are deflated.

15. The transverse bone transport system according to claim 1, further comprising a power source for powering said distraction device, said power source comprising one or more batteries housed in an inner compartment of the base plate.

16. The transverse bone transport system according to claim 1, further comprising a power source for powering said distraction device, said power source comprising one or more receiving coils housed in an inner compartment of the base plate and configured to receive power from an external primary coil.

17. Transverse bone transport method, comprising the steps of:
cutting the skin and subcutaneous tissues of a patient to access a bone site;
implanting a base plate with at least an aperture on the bone site;
performing an osteotomy to detach a bone transport segment from the bone site;
anchoring the bone transport segment to a movable piece attached to the base plate;
closing the incision;
after the incision is closed, operating a distraction device configured to displace the movable piece with respect to the base plate.

18. Transverse bone transport method according to claim 17, wherein the movable piece is displaced in a direction which is substantially orthogonal to the base plate.

19. Transverse bone transport method according to claim 17, wherein the base plate is implanted by means of cannulated, self-tapping bone screws.

20. Transverse bone transport method according to claim 17, wherein the osteotomy is performed by a laser cutting system.

21. Transverse bone transport method according to claim 17, wherein during surgery a flexible tube for connecting external pump units to inflatable bellows interposed between the base plate and the movable piece is routed through the incision.
